(19) United States
(12) Patent Application Publication
(10) Pub. No.: US 2022/0028646 A1
(43) Pub. Date: Jan. 27, 2022

(54) COMPACT DEVICE FOR PULSED ELECTRIC FIELDS IN A TREATMENT CELL

(71) Applicant: Deutsches Institut für Lebensmitteltechnik e.V., Quakenbruck (DE)

(72) Inventors: Tobias Kleemann, Quakenbruck (DE); Johann Isaak, Quakenbruck (DE); Martin Stute, Quakenbruck (DE)

(21) Appl. No.: 17/382,772

(22) Filed: Jul. 22, 2021

(30) Foreign Application Priority Data
Jul. 23, 2020 (DE) 102020209334.2

(51) Int. Cl. H01J 37/04; H01J 37/248; H01J 37/20; H01J 37/16

(52) U.S. Cl. CPC H01J 37/04; H01J 37/248; A23L 3/32; H01J 37/16; H01J 37/20

(57) ABSTRACT

The invention describes a compact device having, in a housing, a chamber which is closable by a flap, one wall of which chamber having at least one first guidance element and at least one first fixation element, as well as two spaced-apart first contact elements which are fixedly connected to electrical connectors of a generator by electrical leads. Further, the device has at least one carrier having at least one second guidance element which, when the carrier is arranged against the wall, engages the first guidance elements and aligns the carrier in a predetermined position with respect to the wall. The carrier has at least one second fixation element that is aligned to match with the at least one first fixation element upon engagement of the first and second guidance elements, such that, when the first and second guidance elements are engaged, the first and second fixation elements are aligned to match one another and can be secured to one another.

### PRIORITY CLAIM

The present application claims priority of the German patent application 10 2020 209 334.2, filed on 23 Jul. 2020.

### FIELD OF THE INVENTION

The present invention relates to a compact device for generating pulsed electric fields between electrodes in a treatment cell in order to apply pulsed electric fields to a medium, liquid or solid, preferably aqueous, which medium is contained in the treatment cell.

### BACKGROUND

DE 10 2009 034 707 B4 for the treatment of liquid media by pulsed electric fields describes two electrodes arranged at a distance from one another in an electrically non-conductive housing, wherein one of the electrodes forms a conduit for feeding the medium between the electrodes.

WO 2018/138361 A1 describes a conveyor belt having two electrodes standing laterally above the conveyor belt, which electrodes can be connected to a voltage supply by solid busbars.

U.S. Pat. No. 5,549,041 describes a PEF device having a container to be fixed to a frame, the walls of which container form electrodes which are pressed against cross-sectional openings of a chamber by spring force.

Maghadam et al., Organic Process R & D, 2001 for chemical syntheses describes a microreactor system having a reaction chamber that is embedded in a plate stack which can be exchangeably introduced into a closable chamber.

US 2008/0063579 A1 describes a device for keeping foods fresh by applying an electric field to the food, wherein the electric field is generated by flexible electrodes.

US 2004/0037736 A1 describes device and process for sterilizing medicinal products in a chamber by double plasma discharge.

### SUMMARY OF THE INVENTION

A preferred embodiment is a compact device for the treatment of media with pulsed electric fields, which device allows for the treatment cell including electrodes to be exchanged with simple handling. In the device, it should preferably be possible to establish the electrical connection of the electrodes to a generator capable of generating electrical pulses without requiring a skilled electrician to install electrical leads. The compact device has the advantage that a simple exchange of the treatment cell containing electrodes is possible and also, during the exchange, electrical leads that can conduct electrical pulses are connected to the electrodes in an operationally reliable manner.

### BRIEF DESCRIPTION OF THE DRAWINGS

Preferred embodiments are described in more detail with reference to the figures, which show in

FIG. 1 a general view of an embodiment of the device,

FIG. 2 a top view of a section of an embodiment of the device,

FIG. 3 a sectional side view of the embodiment of FIG. 1,

FIG. 4 a further embodiment of a section of the device in top view,

FIG. 5 the embodiment of FIG. 3 in sectional side view, and in

FIG. 6 an embodiment of the first and second contact elements.

In the figures, identical reference numerals denote functionally identical elements.

### DETAILED DESCRIPTION OF THE PREFERRED EMBODIMENTS

A preferred compact device has, in a housing, a chamber which is closable by a flap, one wall of which chamber has at least one first guidance element, preferably two first guidance elements, and at least one first fixation element, as well as two spaced-apart first contact elements which are fixedly connected to electrical connectors of a generator by electrical leads. Further, the device includes at least one carrier, preferably at least two carriers, which can e.g. be plate-shaped and is also referred to as a carrier plate. The carrier includes at least one second guidance element, preferably two second guidance elements, which engage with the first guidance elements when the carrier is arranged against the wall and which align the carrier into a predetermined position relative to the wall. The carrier has at least one second fixation element that is aligned to engage with the at least one first fixation element when the first and second guidance elements are engaged, such that the first and second fixation elements are aligned to engage with one another and can be fixed to one another when the first and second guidance elements are engaged.

Optionally, the first guidance elements and first fixation elements can each be formed by one component, and the second guidance elements and second fixation elements can correspondingly each be formed by one common component. Preferably, the first guidance elements and the first fixation elements are formed by respective individual components which are fixed to the wall, and the second guidance elements and the second fixation elements are each formed by individual components which are arranged on the carrier, fixed or movable.

Preferably, the first guidance elements protrude beyond the wall and the second guidance elements are recesses on or in the carrier that are movable over the first guidance elements. The first guidance elements can e.g. be bolts that protrude over the wall and the second guidance elements can be recesses in the carrier which are circumferentially closed or which are open to the edge of the carrier and which can be lifted and/or pushed over the bolts.

The first fixation elements can be internal threads which are attached in the wall, and the second fixation elements can be screws which are rotatably guided on the carrier.

In an embodiment in which the first guidance elements and first fixation elements are formed by one component, such a first component can be formed by a threaded bolt that protrudes over the wall, and the second guidance elements can be recesses formed in the carrier, each matching the threaded bolts, and the second fixation elements can be nuts which can be screwed over the threaded bolts.

The first guidance elements and first fixation elements arranged in the wall, and the second guidance elements and second fixation elements arranged in or on the carrier in a matching manner thereto have the advantage that a carrier can be arranged and fixed to the wall in a simple manner and matching to a predetermined arrangement.

The carrier has at least two second contact elements which are arranged to match with the first contact elements when the guidance elements are engaged and when the carrier is arranged against the wall. The first contact elements preferably extend exclusively behind the first surface of the wall, which first surface faces the chamber, respectively the first contact elements are arranged exclusively behind the first surface of the wall, which first surface faces the chamber, such that the first contact elements preferably do not protrude over the first surface of the wall, which first surface faces the chamber.

The second contact elements are fixed to the carrier in positions aligned to match with the first contact elements and engage with them when, upon engagement of the first and second guidance elements with one another, the carrier is arranged on the wall. The second contact elements protrude over the second surface of the carrier, which first surface faces the wall when the carrier is arranged on the wall.

A treatment cell for medium is attached to the first surface of the carrier, which treatment cell includes at least two electrodes, insulated or electrically conductive, each forming part of the treatment cell, in order to deliver electric pulses to the medium between them, or to generate pulsed electric fields in the medium between them. The electrodes are connected to the second contact elements by leads. Preferably, the carrier consists of insulating material, e.g. non-conductive plastic, at least in the area in which the leads are arranged between the second contact elements and the electrodes and in the region in which the electrodes of the treatment cell are arranged. Further preferably, the carrier consists entirely of insulating material.

Further preferably, the wall, more preferably all walls limiting the chamber, consists of insulating material. Preferably, the flap consists of transparent insulating material, e.g. glass or plastic, e.g. polycarbonate or polyacrylate. The flap can be pivotably or slidably guided on the housing, and it can be single-pieced or multi-pieced.

When the first and second guidance elements are arranged at one another, electrical leads from the connectors of the generator to the electrodes are established by the second contact elements being aligned with and engaging the first contact elements. Since the electrical leads from the electrodes to second contact elements are fixed and the electrical leads from the first contact elements to connectors of the generator are fixed, the device is set up to electrically connect the electrodes of the treatment cell to the generator solely by the connection or engagement of the first and second contact elements. This does not require manipulation of the leads themselves. Thus, the first contact elements with the second contact elements form the only separable connection in the electrical leads between the electrodes and the generator, while electrical leads fixedly connect the electrodes to the second contact elements, and electrical leads fixedly connect the first contact elements to the connectors of the generator. These fixed connections can be formed in that the leads are non-detachably connected to the electrodes and to the second contact elements, e.g. by sheared screws or by welded or soldered connections, connected to the electrodes at one end and to the second contact elements at the other end. Optionally, the leads between the first contact elements and the generator are also non-detachably connected at one end to the first contact elements and at the other end to the connectors of the generator. The generator is preferably a high voltage pulse generator. The connected leads can e.g., be hard-wired.

The arrangement according to the invention of the first contact elements behind the first surface of the wall, which first surface faces the chamber, prevents an accidental contacting of the first contact elements.

Optionally, two or more electrodes of the same polarity can be attached to or within the treatment cell, and the electrodes of the same polarity can be connected to the same second contact element by a common lead or a plurality of individual leads.

Preferably, the device is present in combination with at least one additional carrier, or the device has at least two carriers whose second fixation elements, second guidance elements and second contact elements have the same arrangement. The at least two carriers can be arranged alternatingly on the wall by the guidance elements and fixation elements, wherein the guidance elements align the second contact elements in each case to match the first contact elements. Accordingly, the device is preferably present in combination with at least two carriers which each have the same arrangement of their second fixation elements, second guidance elements and second contact elements, such that these are arranged to match the first fixation elements, first guidance elements and first contact elements.

In the case of one carrier, preferably in the case of at least two carriers, each carrier can e.g. have a treatment cell which is closed in itself for a batch-wise electrical loading of a medium contained in the treatment cell, or can have a treatment cell having an inlet and an outlet for medium for an electrical loading of the medium while the medium is flowing through the treatment cell.

Optionally, the flap has recesses through which at least one first conduit for feeding medium into the treatment cell, preferably at least one second conduit for draining medium from the treatment cell, can be arranged.

Preferably, between the flap and the housing, a safety switch is mounted which is set up to connect the generator to a power connection only when the flap is closed over the chamber, and/or to disconnect the generator from a power connection when the flap is in the open position.

A generator can be arranged in a separate generator housing that is connected to the housing, e.g., at abutting outer housing walls. Preferably, the generator is arranged within the one housing in which the chamber is also arranged. This embodiment has the advantage that the generator and the electrical leads, which can connect the connectors of the generator to the electrodes, extend entirely within a common housing. Preferably, the housing has a control for the generator, e.g. for setting parameters of the electrical pulses.

The electrical leads from the connectors of the generator to the first contact elements and/or the electrical leads from the second contact elements to the electrodes of the treatment cell can be single-core leads or solid conductors, each optionally with an external shield.

Further preferably, the treatment cell has sensors that can be connected to the treatment cell directly or optionally through conduits for medium. Preferably, the sensors include at least one temperature sensor, at least one pressure sensor and/or at least one conductivity sensor, each in contact with the interior of the treatment cell. Therein, the housing has measurement electronics which is set up to receive and evaluate sensor signals. The measurement electronics is contained in the housing and preferably has a display that is accessible at the housing. The display can be formed in the form of a transmitter unit that is set up to transmit sensor signals from the housing, e.g., in the form of radio signals. Such sensor signals sent e.g. in the form of radio signals can e.g. be received by a receiver set up for this purpose, which can be distanced from the housing.

Further optionally, the measurement electronics is connected to the control for the generator and the control is set up to control the generator in dependence on signals from the measurement electronics.

Preferably, each carrier on its first surface or on its second surface has an optical identifier, e.g. a bar code, and an optical detector is arranged on or in the chamber, e.g. on or in the wall against which the carrier is arranged, in a position opposite to which the optical identifier is arranged upon engagement of the guidance elements of the carrier and the wall, optionally upon fixation of the first and second fixation elements to one another. Preferably, the optical detector is set up to transmit signals to the generator, which signals are picked up by the detector based on the optical identifier. In this embodiment, it is preferred that the optical identifier is specific for the electrodes and/or for the type of treatment cell, e.g. closed treatment cell or with feed line and/or drain line for medium from the treatment cell, so that the signal picked up by the detector serves as an identification of the treatment cell. Further optionally, the device is set up to direct the signal as an identification of the carrier to the generator, and the device is set up to control the generator depending on the signal, e.g. to preset maximum values for parameters of the electrical pulses.

It has shown that the optical identification at the carrier and an optical detector, e.g. on or in the wall of the chamber, has the advantage that the identification of the carrier can occur without impairment by electric fields and therefore in an operationally reliable manner. An optical detector can be an array of photosensors or an optical camera.

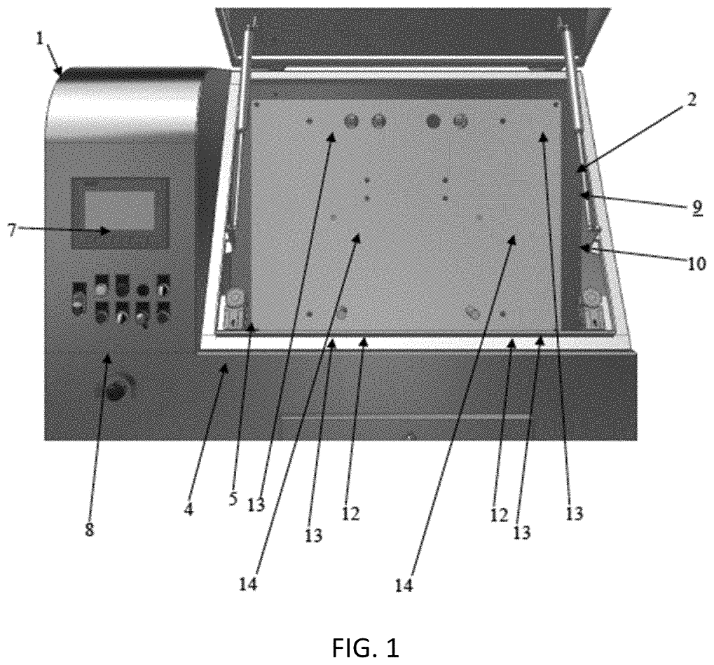

FIG. 1 shows, according to the preferred embodiment, a housing 1 in which both a generator for generating electrical pulses and a chamber 2 are integrated, which chamber 2 can be closed by a flap 3 that is pivotably hinged to the housing 1. To the housing 1 in the area of the frame 4 on which the flap 3 rests in the closed position, a safety switch 5 is attached which is switched by its counterpart 6 on the flap 3 and which, when the flap 3 is in the closed position, generates a signal which allows the generator to be connected to its power connection and which, when the flap 3 is in the open position, disconnects the generator from its power connection. In the embodiment shown here, the housing 1 has a display 7 showing e.g. parameters of the electrical pulses generated by the generator and optionally showing parameters recorded by sensors, and has actuating switches 8 for the generator. The rear wall 10 of the chamber 2 is prepared to receive a carrier 20 and has two first guidance elements 12 in the form of bolts projecting over the wall 10. Separate from the first guidance elements, first fixation elements 13 in the form of threaded holes are arranged in the wall 10. Further, in the wall 10 two first contact elements 14 are arranged, which are sunk-in in the wall and only behind the first surface 9 of the wall 10 extend into the housing 1, which first surface 9 faces the chamber 2.

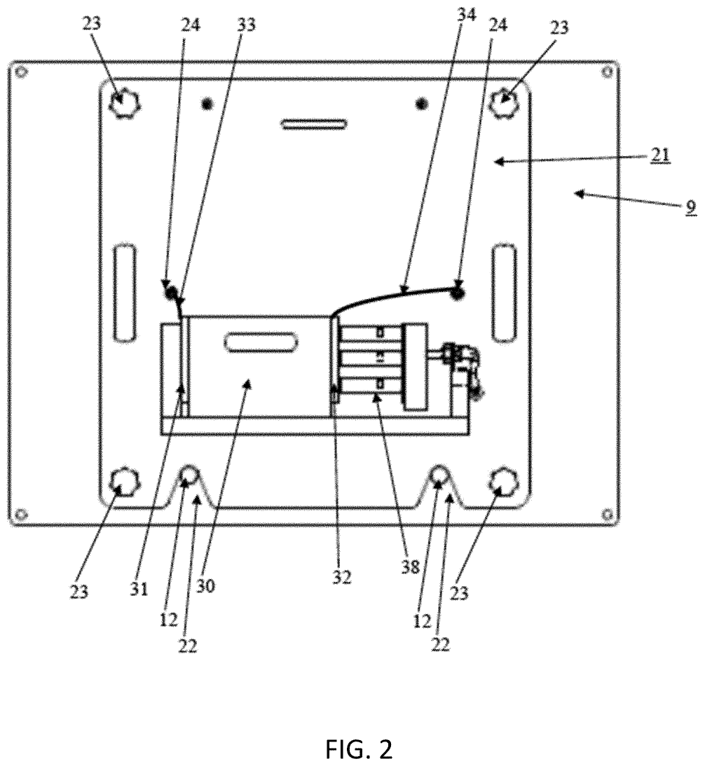

FIG. 2 shows, in top view onto the wall 10, a carrier 20 arranged thereon, the first surface 21 of which lies opposite to the wall 10. Second guidance elements 22 in the form of recesses, which are slidable along the first guidance elements 12 and are positioned thereon, are arranged on the carrier 20. The second fixation elements 23 arranged on the carrier 20 are arranged to match the first fixation elements 13 upon engagement of the first and second guidance elements 12, 22. On the carrier 20, two second contact elements 24 are arranged which protrude over the second surface 25 of the carrier 20, which second surface 25 faces the wall 10, which second contact elements 24 upon engagement of the first and second guidance elements 12, 22 are aligned to match with the first contact elements 14 and engage with these. Attached to the first surface 21 of the carrier 20 is a treatment cell 30 having at least two electrodes 31, 32. The electrodes 31, 32 are connected to one of the second contact elements 24, each by separate electrical leads 33, 34.

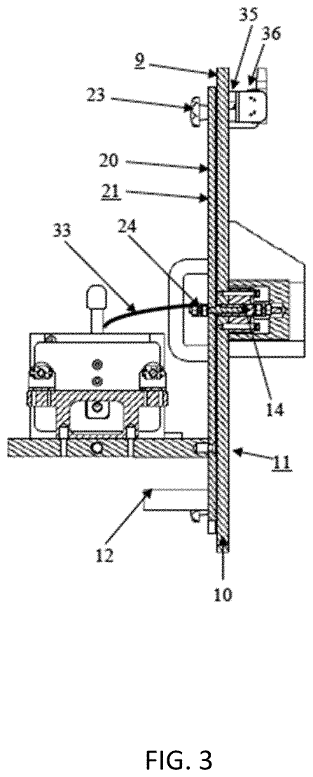

FIG. 3 in sectional view shows the section of FIG. 2, rotated by 90°. According to one embodiment, on the second surface 11 of the wall 10, opposite to its first surface 9, an optical identifier 35 for the carrier or resp. for the treatment cell 30 mounted thereon is attached, and an optical detector 36 lies over the optical identifier 35 when the carrier 20 is arranged against the wall 10. The detector 36 is set up to transmit a signal for the optical identifier 35 to the generator 37 in order to control the generator 37 in dependence from this signal.

The exemplary treatment cell 30 shown in FIG. 2 and FIG. 3 is arranged in a clamping frame 38 with which the electrodes 31, 32 can be clamped towards one another, such that the electrodes 31, 32 can be arranged at different distances depending on the treatment cell 30. This treatment cell 30 can be used for batch-wise treatment of a medium without a connected conduit for medium.

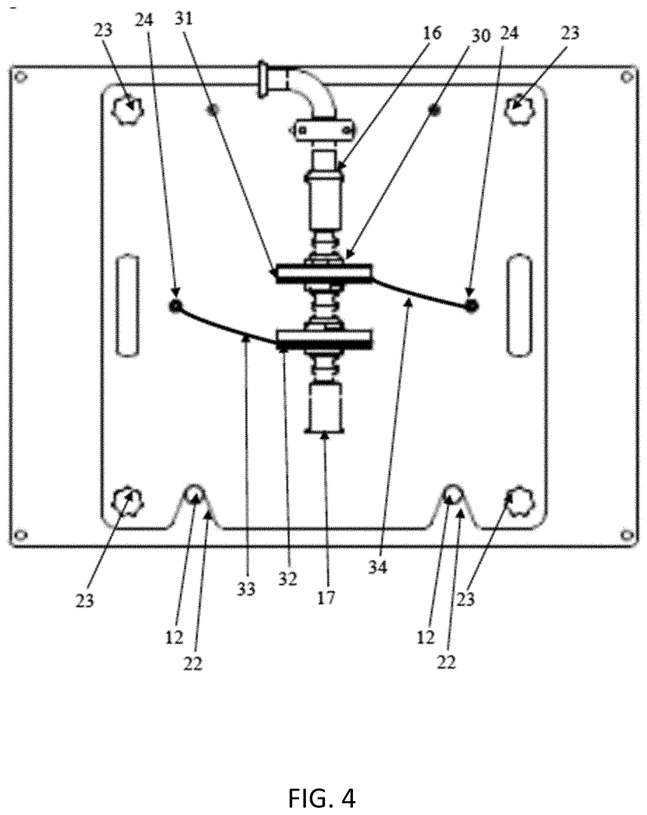

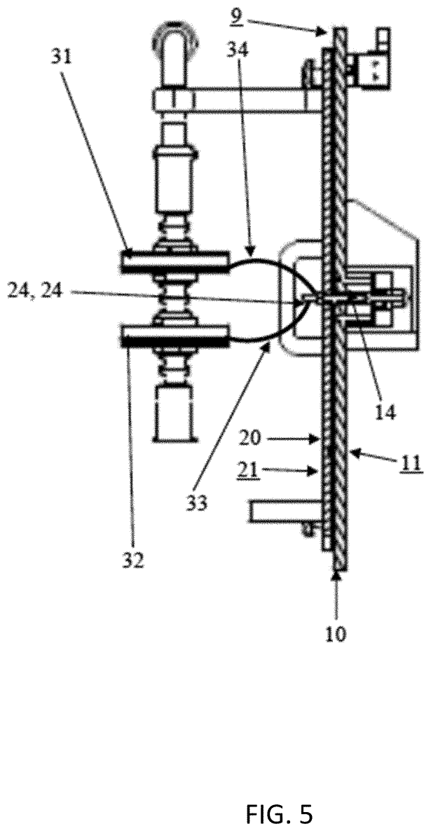

FIG. 4 shows, in top view onto the wall 10, another embodiment of a chamber 2, and FIG. 5 shows this embodiment in sectional view, rotated by 90°. The wall 10 is the same as the wall 10 shown in FIG. 2 and FIG. 3, with the first guidance elements 12 and first fixation elements 13, as well as first contact elements 14, arranged thereon. The carrier 20 shown here has the second guidance elements 22, the second fixation elements 23 and the second contact elements 24 in the same positions as the carrier 20 of FIG. 2 and FIG. 3. Therefore, the carriers 20 can be arranged alternatingly on the wall 10, with their second guidance elements 22 along the first guidance elements 12, can be fixed there by their second fixation elements 23, wherein the second contact elements 24 of the carriers 20 each engage with the same first contact elements 14 of the wall 10, and electrically connect the electrodes 31, 32 to the generator 37.

In the embodiment of FIG. 4, the treatment cell is suitable to generate pulsed electric fields while medium flows along the electrodes. For this purpose, the treatment cell has a first conduit 16 for feeding medium to the treatment cell 30 and preferably has at least one second conduit 17 for draining medium from the treatment cell.

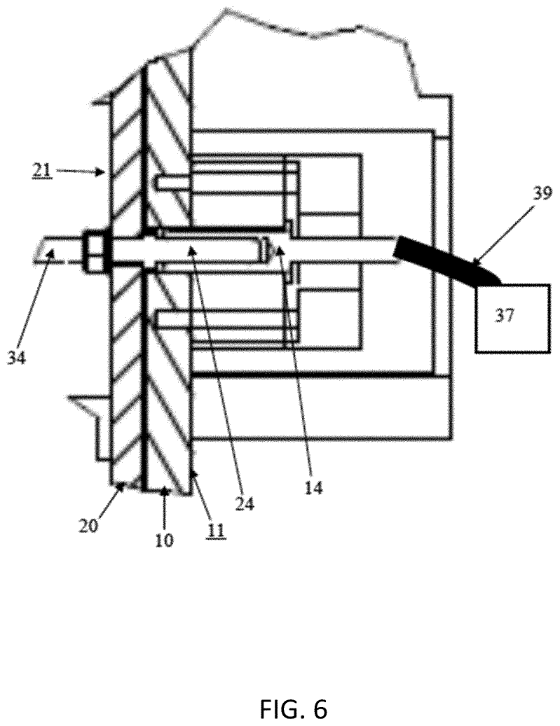

FIG. 6 in sectional view shows a preferred embodiment of a first contact element 14 in engagement with a second contact element 24. Therein, the first contact element 14 extends only in and/or behind the wall 10 from its second surface 11, which lies opposite to its first surface 9 and to the carrier 20 arranged thereon. The second contact element 14 has a recess 15 which is electrically conductive, e.g. a boring in metal. The second contact element 24 protrudes over the carrier 20 and into the recess 14 of the first contact element 14. Each first contact element 14 is fixedly connected to a connector of the generator 37 by an electric lead 39.

### REFERENCE SIGNS

1 housing
2 chamber
3 flap
4 frame
5 safety switch
6 counterpart of the safety switch
7 display
8 actuating switch
9 first surface of the wall
10 wall
11 second surface of the wall
12 first guidance element
13 first fixation element
14 first contact element
15 recess
16 first conduit for medium
17 second conduit for medium
20 carrier
21 first surface of the carrier
22 second guidance elements on the carrier
23 second fixation elements on the carrier
24 second contact element
25 second surface of the carrier
15 recess of the first contact element
30 treatment cell
31, 32 electrode
33, 34 electric lead
35 optical identifier
36 optical detector
37 generator
38 clamping frame
39 electric lead

1. Device for the treatment of a medium by pulsed electric fields, the device having a treatment cell which has at least two electrodes which are connected by electric leads to connectors of a generator for electric pulses, wherein the treatment cell is arranged in a chamber in a housing, which chamber is closable by a flap, wherein the treatment cell is attached to a carrier and the chamber has a wall on which at least one first guidance element is arranged that can be brought into engagement with at least one second guidance element which is arranged on the carrier, at least one first fixation element is arranged on the wall, to which first fixation element upon engagement of the first and second guidance elements at least one second fixation element can be fixed, which second fixation element is arranged on the carrier, and in that at least two first contact elements are arranged on the wall, into each of which first contact elements respective second contact elements arranged on the carrier engage upon engagement of the first and second guidance elements, and the electrodes are fixedly connected to the second contact elements by electrical leads, and the first contact elements are fixedly connected to the connectors of the generator by electrical leads.

2. Device according to claim 1, wherein the first contact elements extend into the housing only within the wall and/or from the second surface of the wall, which second surface lies opposite to the carrier, and in that the second contact elements protrude over the second surface of the carrier, which second surface faces the wall.

3. Device according to claim 1, wherein at least one additional carrier whose second fixation elements, second guidance elements and second contact elements of each carrier have the same arrangement.

4. Device according to claim 1, wherein the electrodes are fixedly connected to the second contact elements by non-detachable electrical leads.

5. Device according to claim 1, wherein the at least one first guidance element protrudes over the wall and the at least one second guidance element is formed by a recess in the carrier.

6. Device according to claim 1, wherein the carrier and the wall each consist of insulating material.

7. Device according to claim 1, wherein one first guidance element and one first fixation element each are formed by one common component.

8. Device according to claim 1, wherein the generator and the chamber are arranged in a common housing.

9. Device according to claim 1, wherein between the flap and the housing, a safety switch is mounted which is set up to generate a signal that the generator is to be connected to a power connection only when the flap is closed over the chamber.
